(19) United States
(12) Patent Application Publication
(10) Pub. No.: US 2005/0256566 A1
(43) Pub. Date: Nov. 17, 2005

(54) Apparatus and method for improving ventricular function

(76) Inventor: Shlomo Gabbay, Short Hills, NJ (US)

Correspondence Address: TAROLLI, SUNDHEIM, COVELL & TUMMINO L.L.P., 526 SUPERIOR AVENUE, SUITE 1111, CLEVEVLAND, OH, 44114, US

(21) Appl. No.: 11/126,036

(22) Filed: May 10, 2005

Related U.S. Application Data
(63) Continuation-in-part of application No. 10/837,944, filed on May 3, 2004.

(51) Int. Cl. A61F 2/24; A61N 1/362

(52) U.S. Cl. 623/2.1; 623/2.14; 623/2.12; 623/2.28

(57) ABSTRACT

An approach is disclosed for improving ventricular function of a patient's heart. In one example, an implantable apparatus includes an inflow conduit having first and second ends spaced apart from each other by a sidewall portion. An inflow valve is operatively associated with the inflow conduit to provide for substantially unidirectional flow of blood through the inflow conduit from the first end to the second end of the inflow conduit. A pouch has an interior chamber that defines a volume. The inflow conduit is in fluid communication with the interior chamber of the pouch. An outflow conduit is in fluid communication with the interior chamber of the pouch to permit substantially free flow of fluid from the interior chamber of the pouch and into the outflow conduit, which terminates in an outflow annulus spaced from the pouch.

### CROSS REFERENCE TO RELATED APPLICATION

The present application is a continuation-in-part of U.S. Patent Application Ser. No. 10/837,944, which was filed on May 3, 2004, and entitled SYSTEM AND METHOD FOR IMPROVING VENTRICULAR FUNCTION.

### TECHNICAL FIELD

The present invention relates to the heart, and more particularly to a system and method for improving ventricular function.

### BACKGROUND

Dilated cardiomyopathy is a condition of the heart in which ventricles one or more become too large. Dilated cardiomyopathy occurs as a consequence of many different disease processes that impair myocardial function, such as coronary artery disease and hypertension. As a consequence of the left ventricle enlarging, for example, the ventricles do not contract with as much strength, and cardiac output is diminished. The resulting increase in pulmonary venous pressure and reduction in cardiac output can lead to congestive heart failure. Dilated cardiomyopathy can also result in enlargement of the mitral annulus and left ventricular cavity, which further produces mitral valvular insufficiency. This in turn, causes volume overload that exacerbates the myopathy, often leading to progressive enlargement and worsening regurgitation of the mitral valve.

A dilated ventricle requires more energy to pump the same amount of blood as compared to the heart of normal size. The relationship between cardiac anatomy and pressure has been quantified by La Place's law. Generally, La Place's law describes the relationship between the tension in the walls as a function of the transmural pressure difference, the radius, and the thickness of a vessel wall, as follows:
T=(P*R)/M, which solving for P reduces to: 1.
P=(T*M)/R 2.
where T is the tension in the walls, P is the pressure difference across the wall, R is the radius of the cylinder, and M is the thickness of the wall.
   Therefore, to create the same pressure (P) during ejection of the blood, much larger wall tension (T) has to be developed by increase exertion of the cardiac muscle. Such pressure further is inversely proportional to the radius of the cylinder (e.g., the ventricle).

Various treatments exist for patients having dilated cardiomyopathy. One approach is to perform a heart transplant procedure. This is an extraordinary measure, usually implemented as a last resort due to the risks involved.

Another approach employs a surgical procedure, called ventricular remodeling, to improve the function of dilated, failing hearts. Ventricular remodeling (sometimes referred to as the Batista procedure) involves removing a viable portion of the enlarged left ventricle and repairing the resultant mitral regurgitation with a valve ring. This procedure attempts to augment systemic blood flow through improvement in the mechanical function of the left ventricle by restoring its chamber to optimal size. In most cases, partial left ventriculectomy is accompanied by mitral valve repair. With respect to La Place's law, a goal of ventriculectomy is to reduce the radius so that more pressure can be generated with less energy and less stress exertion by the patient's cardiac muscle.

### SUMMARY

One aspect of the present invention provides a system for improving operation of a heart.

According to one aspect of the present invention, an implantable apparatus includes an inflow conduit having first and second ends spaced apart from each other by a sidewall portion. An inflow valve is operatively associated with the inflow conduit to provide for substantially unidirectional flow of blood through the inflow conduit from the first end to the second end of the inflow conduit. A pouch has an interior chamber that defines a volume. The inflow conduit is in fluid communication with the interior chamber of the pouch. An outflow conduit is in fluid communication with the interior chamber of the pouch to permit substantially free flow of fluid from the interior chamber of the pouch and into the outflow conduit, which terminates in an outflow annulus spaced from the pouch.

Another aspect of the present invention provides an apparatus for improving ventricular function. The apparatus includes means for limiting a volume of blood received within an enlarged ventricle of the patient's heart; means for providing for substantially unidirectional flow of blood into the means for limiting; means for providing a path for flow of blood from within the means for limiting and into an aorta of the patient's heart; and means, located within the means for providing a path, for providing for substantially unidirectional flow of blood out of the means for limiting and into the aorta.

Yet another aspect of the present invention provides a method for improving ventricular function of a heart. The method includes implanting a pouch in a ventricle of the heart, the pouch including an interior chamber that defines a volume. An inflow valve is mounted at a mitral position of the heart, the inflow valve being in fluid communication with the interior chamber of the pouch to provide for substantially unidirectional flow of blood from an atrium of the heart through the inflow valve and into the interior chamber of the implanted pouch. An outflow conduit, which is in fluid communications with the interior chamber of the implanted pouch, is attached near an aortic annulus to provide for substantially unidirectional flow of blood from the interior chamber of the pouch and into the aorta of the heart. By way of further example, blood can be removed from a space in the ventricle between the pouch and surrounding cardiac tissue to facilitate self-remodeling of the heart. For instance, one or more conduits can be attached between the ventricle and the atrium to provide a path for flow of blood from the space in the ventricle to the atrium.

### BRIEF DESCRIPTION OF THE DRAWINGS

FIG. 1 depicts an example of a system for improving ventricular function according to an aspect of the present invention.

FIG. 2 depicts an example of another system for improving ventricular function according to an aspect of the present invention.

FIG. 3 is a cross-sectional view of a heart illustrating a condition of dilated cardiomyopathy.

FIG. 4 illustrates a system for improving ventricular function implanted in a left ventricle according to an aspect of the present invention.

FIG. 5 depicts an example of another system for improving ventricular operation implanted in a ventricle in combination with an aortic valve according to an aspect of the present invention.

FIG. 6 depicts an example of another system for improving ventricular function implanted in a ventricle in combination with an aortic valve according to an aspect of the present invention.

FIG. 7 depicts an example of an apparatus that can be utilized to improve ventricular function according to an aspect of the present invention.

FIG. 8 depicts another view of the apparatus of FIG. 7 according to an aspect of the present invention.

FIG. 9 depicts an assembly view of an example of another apparatus that can be utilized to improve ventricular function according to an aspect of the present invention.

FIG. 10 depicts an example the assembled apparatus of FIG. 9 according to an aspect of the present invention.

FIG. 11 depicts an example of another system for improving ventricular function implanted in a ventricle according to an aspect of the present invention.

### DETAILED DESCRIPTION

FIG. 1 depicts an example of a system 10 for improving ventricular function of a heart. The system 10 includes an enclosure or pouch 12 that is dimensioned and configured to simulate at least a portion of a normal heart chamber. As used herein, the term “pouch” refers to a pocket or saclike structure having an interior chamber that defines a volume that can hold fluid, such as blood, therein. The particular shape or configuration of the pouch can vary from that shown and described herein without departing from the spirit and scope of the present invention. The pouch 12 includes an inflow annulus 14 spaced apart from a distal closed end 16 by a generally cylindrical sidewall 18. In the example of FIG. 1, the sidewall 18 of the pouch 12 has a generally pear-shaped contour, in which the portion of the sidewall 18 proximal the inflow annulus 14 has a reduced diameter relative to an intermediate portion thereof proximal the distal end 16.

A generally cylindrical outflow portion (e.g., a tubular branch) 20 extends from the sidewall 18 of the enclosure 12. The outflow portion 20 extends longitudinally from a first end 22 and terminates in an outflow end 24 that is spaced apart from the first end 22 by a generally cylindrical sidewall thereof. The first end 22 can be attached to the sidewall 18. For instance, the first end 22 can be connected to the sidewall 18 via a continuous suture to couple the outflow portion 20 with the sidewall portion such that fluid (e.g., blood) can flow from the chamber defined by the pouch 12 through the outflow portion 20. Alternatively, the first end 22 can be formed integral with the sidewall 18.

The system 10 also includes a valve 26 operatively associated with the inflow annulus 14. The valve 26 is configured to provide for substantially unidirectional flow of blood through the valve into the chamber defined by the pouch 12. For example, when the system 10 is mounted in a left ventricle, blood will flow from the left atrium through the valve 26 and into the chamber, which defines a volume of the pouch 12. The pouch, when implanted in the ventricle, thus provides means for limiting a volume of blood received within an enlarged ventricle of the patient's heart. When the outflow end is located in a patient's aorta, the outflow portion 20 also corresponds to means for providing a path for the flow of blood from within the pouch and into the aorta.

Those skilled in the art will understand and appreciate that practically any type of prosthetic valve 26 can be utilized to provide for the unidirectional flow of blood into the chamber. For example, the valve 26 can be implemented as a mechanical heart valve prosthesis (e.g., a disc valve, ball-check valve, bileaflet valve), a biological heart valve prosthesis (homograft, autograft, bovine or porcine pericardial valve), or a bio-mechanical heart valve prosthesis (comprising a combination of mechanical valve and natural tissue materials), any of which can include natural and/or synthetic materials. Additionally, the valve 26 can be a stented valve or an unstented valve.

In the example of FIG. 1, the valve 26 is depicted as a biological heart valve prosthesis that is mounted at the annulus 14, such as by suturing an inflow annulus of the valve 26 to the annulus 14 of the system 10. The valve 26 can include one or more leaflets (typically two or three) or other movable members adapted to provide for desired unidirectional flow of blood through the valve and into the chamber of the pouch 12.

When a biological heart valve prosthesis is utilized to provide the valve 26, the valve typically includes two or more leaflets 30 movable relative to the annulus 14 to provide for the desired unidirectional flow of blood into the pouch 12. The leaflets 30 are mounted for movement within the inflow portion of the pouch 12, namely near the annulus 14. In the illustrated embodiment of FIG. 1, the leaflets 30 are mounted relative to a sidewall valve portion 32 of a previously harvested heart valve, which has been treated to improve its biocompatibility and mounted within a stent. The inflow end of the valve 26 is sutured to the inflow annulus 14 of the pouch 12, such as by sewing (or otherwise affixing) a sewing ring thereof relative to the annulus 14. An outflow end of the valve wall portion 32 of the valve 26 can be sewn by sutures 34 to the sidewall 18 of the pouch 12.

The pouch 12 can be formed of a biological tissue material, such as previously harvested animal pericardium, although other natural tissue materials also can be utilized (e.g., duramatter, collagen, and the like). The pericardium sheet or sheets utilized to form the pouch 12 has opposed interior/exterior side surfaces. According to one aspect of the present invention, the pericardial sheet(s) are oriented so that a rougher of the opposed side surfaces forms the interior sidewall portion of the chamber. The rougher surface facilitates formation of endothelium along the interior of the sidewall 18 thereby improving biocompatibility of the system 10.

By way of further illustration, the pouch 12 may be formed from one or more sheets of a NO-REACT® tissue product, which is commercially available from Shelhigh, Inc., of Millburn, N.J. as well as from distributors worldwide. The NO-REACT® tissue products help improve the biocompatibility of the system 10, thereby mitigating the likelihood of a patient rejecting the system. The NO-REACT® tissue also resists calcification when implanted. Those skilled in the art will appreciate various other materials that could be utilized to form the pouch 12, including collagen impregnated cloth (e.g., Dacron) as well as other biocompatible materials (natural or synthetic). The NO-REACT® tissue products further have been shown to facilitate growth of endothelium after being implanted.

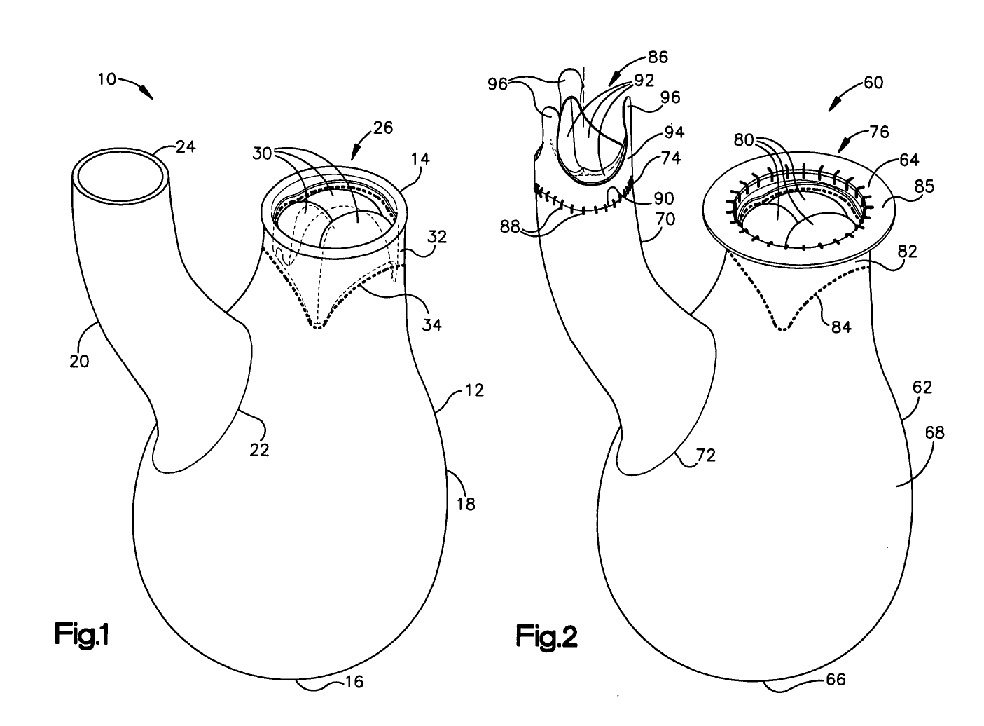

FIG. 2 depicts an example of another system 60 that can be utilized to improve ventricular function according to an aspect of the present invention. The system 60 is substantially similar to that shown and described in FIG. 1. Accordingly, the reference numbers used in FIG. 2 are the same, increased by adding 50, as utilized to identify the corresponding parts previously identified in FIG. 1.

Briefly stated, the system 60 includes a pouch 62 dimensioned and configured to simulate at least a portion of a heart chamber, such as a ventricle. The pouch 62 includes an inflow annulus 64 spaced apart from a closed distal end 66 by a generally cylindrical (e.g., pear-shaped) sidewall 68. A generally cylindrical outflow portion 70 extends from the sidewall 68, which is configured for providing a fluid path from the interior of the pouch 62 to an aorta. The outflow portion 70 can be configured as a length of a generally cylindrical tissue that extends from a first end 72 connected to the sidewall 68 and terminates in a second end spaced 74 apart from the first end.

The system 60 also includes an inflow valve 76 at the inflow annulus 64, which provides for substantially unidirectional flow of blood into the chamber defined by the pouch 62. Various types and configurations of valves could be employed to provide the valve 76, such as mentioned herein. In the example of FIG. 2, the valve is depicted as a biological heart valve prosthesis having a plurality of leaflets 80 positioned for movement relative to an associated sidewall portion 82. An outflow end 84 of the valve 76 is attached at the inflow annulus 64 of the pouch 62 and extends into the pouch. The outflow end 84 can be sutured to the pouch 62. A sewing ring 85 can be provided at the inflow end of the valve 76 to facilitate its attachment at a mitral annulus of a patient's heart.

In the example of FIG. 2, an outflow valve 86 is also mounted at the outflow end 74 of the outflow portion 70. For example, the valve 86 can be attached to the outflow end 74 by sutures 88. While an inflow end 90 of the valve 86 is illustrated as being anastomosed to the inflow end 74 of the outflow portion 70, it will be understood and appreciated that, alternatively, an inflow extension of the valve 86 or the sidewall of the outflow portion 70 can be an overlapping relationship relative to the other. As still another alternative, the valve 86 can be integrally formed with the outflow portion 70.

In the example of FIG. 2, the valve 86 is illustrated as a biological heart valve prosthesis. The valve 86 thus includes a plurality of leaflets 92 positioned for movement within a corresponding sidewall portion 94 of the valve 86 to provide for substantially unidirectional flow of blood axially through the valve 86, as provided from the pouch 62. The valve 86 can be stented or unstented. The plurality of corresponding outflow extensions 96 are positioned at respective commissures of the valve 86 to facilitate its attachment and to maintain the valve at the aortic position of a patient's heart.

While the valve 86 is illustrated as a biological heart valve prosthesis, those skilled in the art will understand and appreciate that any type of valve can be utilized at the outflow annulus 74. By way of example, the valve 86 can be implemented as a mechanical heart valve, a biological heart valve or a bio-mechanical heart valve prosthesis. The valve 86 can be the same or a different type of valve from that utilized for the valve 76. Additionally, while the valve 86 is depicted as attached at the outflow annulus 74, the valve could be attached proximal the first end 72 or any where between the ends 72 and 74. It is to be appreciated that the valve 86 can be attached to the outflow portion 70 (e.g., through the aorta) after the other parts of the system 60 have been implanted.

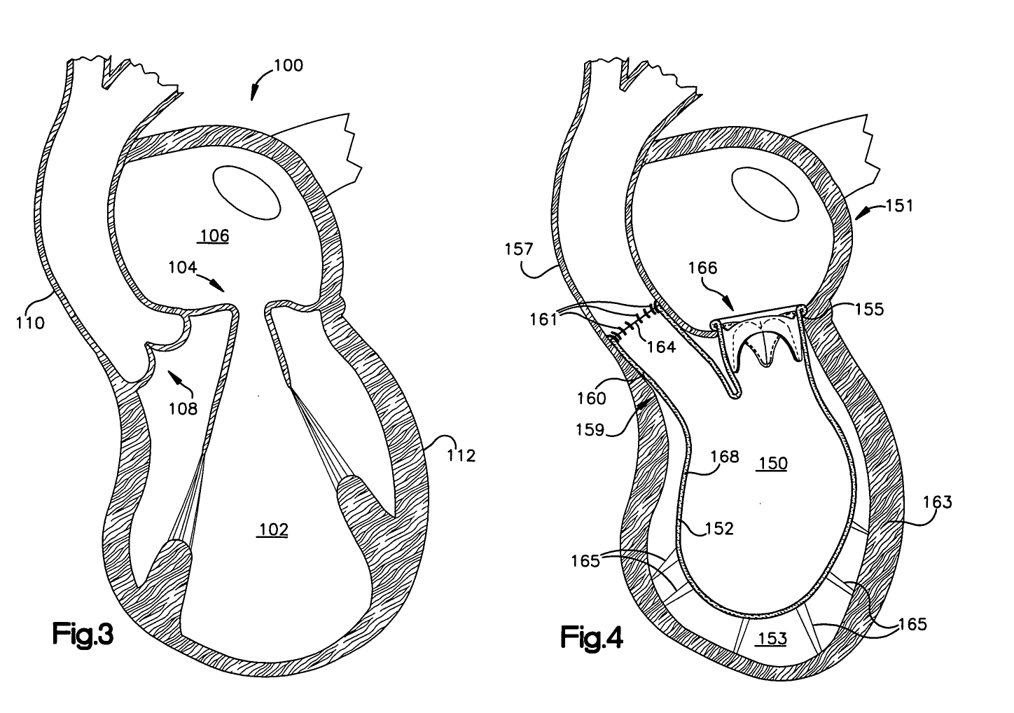

FIG. 3 depicts an example of a heart 100 in which a left ventricle 102 is severely dilated, such as in the case of dilated cardiomyopathy. As a result of the dilated left ventricle 102, a mitral valve 104 can severely prolapse, such that the mitral valve 104 is unable to provide for desired unidirectional flow of blood from the left atrium 106 to the left ventricle 102.

In the example of FIG. 3, the aortic valve 108 appears intact and sufficient, although in many cases, the aortic valve may also be defective. The aortic valve 108, when operating properly, provides for a substantially unidirectional flow of blood from the left ventricle 102 into the aorta 110. As a result of the dilation of the left ventricle 102, however, associated cardiac muscle 112 of the heart 100 is required to expend greater energy to pump the same amount of blood in the absence of such dilation. The extra exertion can be described according to the well-know La Place's law, such as mentioned in the Background section.

FIG. 4 illustrates an example of a system 150 for improving ventricular function that has been implanted in a heart 151. The system 150 is substantially similar to the system shown and described with respect to FIG. 1, and reference numbers, increased by adding 140, refer to corresponding parts of the system 10 previously identified with respect to FIG. 1. Briefly stated, the system 150 includes a pouch 152 dimensioned and configured to simulate at least a portion of a properly functioning ventricle. Thus, by positioning the system 150 in the ventricle 153 of the heart 151, as shown in FIG. 4, ventricular function can be substantially improved (when compared to the dilated heart of FIG. 3). The pouch 152 can be generally pear-shaped extending from a valve 166 attached at a mitral annulus 155 of the heart 151.

A generally cylindrical outflow portion 160 extends from the sidewall 168 of the pouch 152 to fluidly connect the pouch with the aorta 157. As shown, the outflow end of the tubular brands 160 can be attached to the aorta 157 near the aortic annulus 159, such as by sutures 161. Prior to inserting the outflow portion 160 into the aorta 157, the patient's native aortic valve can be removed and the outflow annulus of the outflow portion can be positioned relative to the aortic annulus 159. Alternatively, it may also be possible to connect the outflow portion 160 of the system 150 to the patient's native aortic valve, thereby leaving the patient's valve intact. A more likely scenario, however, is that the aortic valve will be removed and replaced by a heart valve prosthesis. The length of the outflow portion 160 may also but cut to a desired length, and then sutured to the base of the aorta 157. This part of the process can be performed through an incision made in the aorta 157.

The valve 166 thus provides for substantially unidirectional flow of blood into from the atrium into the chamber defined by the pouch 152. Various types and configurations of valves could be employed to provide the valve 166, such as described herein.

By way of further example, prior to implanting the system 150 in the left ventricle 153, the dilated mitral annulus can be forced to a reduced diameter. For instance, the mitral annulus can be reduced by applying a purse-string suture around the mitral annulus and closing the purse-string suture to a desired diameter, such as corresponding to the diameter of the valve 166 that is to be implanted. The annulus of the inflow valve 166 can then be sutured to the mitral annulus 155, such as shown in FIG. 4. The outflow end of the outflow portion 160 further can be sutured to the sidewall of the aorta 157 to maintain the outflow portion at a desired position relative to the aorta (e.g., at the base of the aorta).

The chamber of the pouch 152 implanted in the dilated ventricle 153 simulates the function of a normal ventricle. That is, the pouch 152 operates to limit the volume of blood within the ventricle since the pouch has a reduced cross-section relative to the patient's dilated ventricle. Consistent with La Place's law, blood can be more easily (e.g. less exertion from cardiac muscle 163) pumped from the chamber of the system 150 than from the patient's native dilated ventricle. That is, the system 150 provides a chamber having a reduced volume relative to the volume of the dilated ventricle, such that less energy and reduced contraction by the associated cardiac muscle 163 are required to expel a volume of blood at a suitable pressure from the pouch 152.

Portions of the sidewall of the system 150 further can be secured relative to the cardiac muscle 163, such as by employing strips 165 of a suitable biocompatible tissue to tether various parts of the sidewall 168 relative to the surrounding cardiac muscle. The strips 165 can help hold the pouch 152 in a desired shape relative to the dilated ventricle 153 during contractions of the cardiac muscle 163. After or during implantation, blood and other fluid in the pouch 152 can be removed from around the system 150 to enable the heart 151 to return to a more normal size. In such a situation, the strips 165 of tissue may remain, but typically will become less functional since their tethering function is reduced after the heart returns to a more normal size.

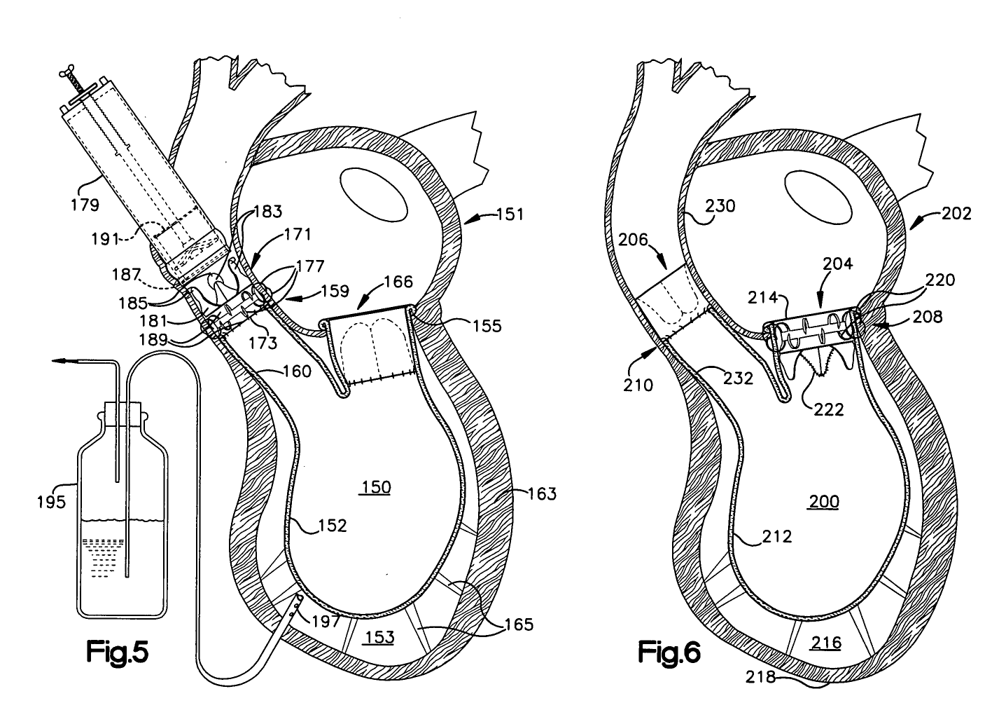

FIG. 5 depicts the system 150 being implanted in combination with an aortic valve 171 according to an aspect of the present invention, in which the same reference numbers refer to the same parts identified with respect to FIG. 4. In FIG. 5, an additional valve 171 is attached at the outflow annulus 164 of the outflow portion 160. As described herein, various types of valves can be employed at the aortic position. FIG. 5 and FIG. 6 provide but two examples of numerous different types of valves that can be utilized.

In the example of FIG. 5, the valve 171 can be implanted at the aortic position according to a generally sutureless method of implantation (“sutureless” meaning that sutures are not required, but sutures can still be used), such as shown and described in co-pending U.S. patent application Ser. No. 10/778,278, which was filed on Feb. 13, 2004, and which is incorporated herein by reference. The outflow valve 171 typically will be implanted after the outflow portion 160 of the system 150 has been attached to the aorta 157 (e.g., by continuous sutures through an opening made in the aorta). Additionally, prior to implanting the valve 171, the patient's own aortic valve or at least calcified portions thereof should be removed.

As shown in FIG. 5, the valve 171 is being implanted through an opening in the patient's aorta 157. The valve 171 includes an inflow end 173 that is positioned at the aortic annulus 159, with an outflow end 175 of the prosthesis extending into the aorta 157. As mentioned above, the implantation can be considered sutureless since the valve 171 includes spikes or other projections 177 that extend radially outwardly from the exterior part of the valve.

In the example of FIG. 5, the spikes 177 are arranged as sets of fingers that extend arcuately toward each other in substantially opposite directions so as to form a clamp-like structure. Additionally, the respective sets of opposing fingers can be arranged in a generally circular array circumferentially about a base portion of the valve 171 proximal the inflow 173 end thereof. For example, each adjacent pairs of fingers alternate in first and second axial directions with one another and are spaced circumferentially apart along the base portion of the valve 171. The ends of the spikes 177 can also be sharpened to facilitate their insertion into the tissue at the aortic annulus 159.

The spikes 177 can be constructed of a resilient material, such as a metal or plastic. A generally resilient material should be sufficiently elastic to permit the spikes 177 to be deformed from an original first condition, extending outwardly to form the clamp-like structure, to a second condition. In the second condition, the sets of spikes 177 are oriented substantially linearly and generally parallel with the longitudinal axis of the valve (but in opposite directions relative to the base portion), and be capable of returning substantially to their original first condition. The valve 171 is carried within an implanter 179 that holds the spikes in the second condition to facilitate positioning of the valve at the aortic annulus 159. The implanter can be of the type shown and described in the above-incorporated application Ser. No. 10/778,278, although other types of implanters could also be utilized.

By way of further example, the implanter 179 can be inserted through an incision in the aorta 157, such as part of an aortotomy procedure (e.g., a transverse aortotomy) while the patient is on cardio-pulmonary bypass. The implanter 179 can be employed to position the distal end of the cylindrical member at a desired location relative to the annulus 159. Once at the desired position, the valve can be discharged from the implanter 179, such that an inflow set of spikes 177 return toward their original shape to penetrate into the surrounding tissue at the annulus 159 tissue. After the remaining length of the prosthesis is discharged, an outflow set of the spikes 177 are also released to return toward their original shape to penetrate into the annulus 159 tissue (e.g., the first condition as shown in FIG. 5).

In the implanted position, an outflow portion 181 of the valve 171 thus extends axially into the aorta 157, with the respective sets of spikes 177 cooperating to inhibit axial as well as rotational movement of the valve relative to the aortic annulus 159. Additionally, lobes (or outflow valve extensions) 183 extending from the outflow commissures of the valve can be attached to the sidewall of the aorta 157, such as by sutures 185. By attaching the lobes 183 to the aorta 157, improved valve competence and coaptation can be achieved, and prolapse can be mitigated.

In order to facilitate loading the valve 171 into the implanter 179, the implanter can include a retaining mechanism 187. The retaining mechanism 187 can be in the form of a retaining ring dimensioned and configured to slide along the exterior of the valve 171. In the example of FIG. 5, the implanter includes a guide system 191 operative to move the retaining mechanism 187 for repositioning the spikes 177 to the second condition. A number of connecting elements (e.g., sutures) connect to the retaining mechanism 187, so that the retaining mechanism may move commensurately with axial movement of the guide system 191.

The valve 171 can also include a covering 189 of a biocompatible material connected for movement with the spikes, such as by connected by sutures (not shown). The covering 258 can be implemented as a pair of generally annular sheet (one for the inflow set of spikes and one for the set of outflow spikes) that move as a function of the movement of the spikes 177.

Additionally, to facilitate implantation of the pouch 152 within the ventricle 153, a vacuum assembly or pump 195 can be employed to remove fluid from the patient's dilated ventricle. Those skilled in the art will understand and appreciate various types of pump devices that could be utilized. The pump 195 can include one or more nozzles or other members 197 fluidly connected with the pump for removing the blood from the ventricle 153. By removing the blood from the dilated ventricle 153, self-remodeling of the cardiac muscle to a more normal size is facilitated.

FIG. 6 depicts yet another example of a system 200 implanted for improving ventricular function of a heart 202. The system of FIG. 6 is similar to that shown and described in FIG. 5, but different types and configurations of biological heart valves 204 and 206 are utilized at the mitral annulus 208 and aortic annulus 210, respectively. In the particular example of FIG. 6, a sutureless type of valve 204 is implanted at the mitral annulus 208 and a more conventional type of biological heart valve prosthesis 206 is employed at the aortic annulus 210. While the examples of FIG. 6 depict biological heart valve prostheses being employed at aortic and mitral positions, those skilled in the art will understand and appreciate that other types of valves (e.g., mechanical, biological, bio-mechanical) can also be utilized. That is, as described herein, any type of valve can be provided at either of the position according to an aspect of the present invention, and the valves at the respective positions can be the same or different types of valves.

By way of further example, the dilated, insufficient pulmonic valve (or at least calcified portions) thereof should be removed from the mitral annulus 208 prior to implanting the valve 204. The valve 204 is attached to a pouch 212 configured to simulate a substantially normal ventricle. The pouch is positioned within the ventricle, such as shown in FIG. 6. To attach the valve 204 at the annulus 208, an inflow end 214 of the valve is annularized with respect to the annulus 208. The positioning and implantation of the valve 204 can be implemented employing an implanter, such as described herein with respect to FIG. 5 and the above-incorporated application Ser. No. 10/778,278. In one approach, the system 200, including the valve 204 can be positioned into the ventricle 216 of the heart 202 through an incision made in the apex 218 of the heart 202.

The valve 204 can be substantially the same as the valve 171 shown and described with respect to FIG. 5. Accordingly, details of such valve have been omitted from the description of FIG. 6 for sake of brevity, and since reference can be made to FIG. 5. Once at the desired position, the valve 204 can be discharged from the implanter, such that an the opposed spikes 220 can return to their normal clamp-like condition and penetrate into the annulus 208 tissue. The respective sets of spikes 220 thus cooperate to anchor the valve 204 relative to the annulus 208 (e.g., clamping onto the tissue at the annulus) so as to inhibit axial and rotational movement of the valve.

In the implanted position, an outflow portion 222 of the valve 204 thus extends axially into the chamber defined by the pouch 212, which is located within the ventricle 216. Additionally, the outflow portion 222 of the valve can be sutured or otherwise secured to the sidewall of the pouch 212 proximal the inflow annulus thereof. As described herein, the valve 204 can be stented or unstented.

The outflow valve 206 can be any type of valve, such as a biological valve depicted in FIG. 6. The valve 206 can be implanted through an incision in the aorta 230, such as after the pouch 212 and the valve 204 have been mounted in the heart 202. For instance, the tubular branch 232 extending from the sidewall of the pouch can be secured (e.g., by continuous sutures) to the base of the aorta 230. Then the valve can be positioned at the aortic annulus and implanted to provide for substantially unidirectional flow of blood from the pouch 212 and into the aorta through the valve 206. The incision in the aorta 230 can then be closed in a desired manner.

The interstitial space in the ventricle 216 between the pouch 212 and the cardiac muscle 234 will reduce over time, enabling the heart to self-remodel and function more normally. The remodeling can be facilitated by removing surrounding fluid, such as via suction device, as depicted with respect to FIG. 5. Those skilled in the art will understand and appreciate that any type of valves can be employed at either of the aortic and mitral positions, and that the valves depicted herein are for purposes of illustration and not by way of limitation.

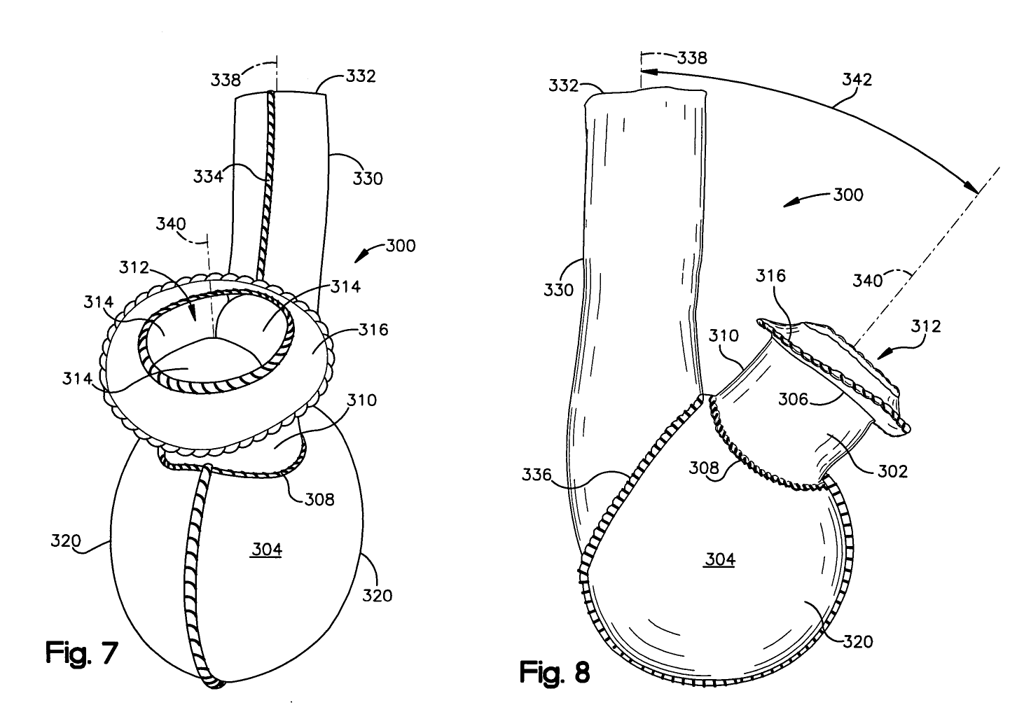

FIGS. 7 and 8 depict another example of an apparatus 300 that can be utilized to improve ventricular function of a patient's heart in accordance with an aspect of the present invention. The apparatus 300 includes an inflow conduit 302 that extends from a pouch 304. In particular, the inflow conduit 302 has first and second ends 306 and 308 spaced apart from each other by a sidewall portion 310. The second end 308 can be attached to the pouch by any suitable means. For example, the second end of the conduit can be anastomosed at a corresponding annulus of the pouch 304, such as by uninterrupted (or continuous) sutures.

An inflow valve 312 is operatively associated with the inflow conduit 302 to provide for substantially unidirectional flow of blood through the inflow conduit from the first end 306 to the second end 308 of the inflow conduit and into an interior chamber of the pouch 304. In the example of FIGS. 7 and 8, the inflow conduit includes the inflow valve 312 located therein. For instance, the sidewall portion 310 can correspond to the valve wall of the inflow valve 312 such that the valve and sidewall portion are integral. As described herein with respect to the preceding examples, any type of heart valve prosthesis can be utilized as the inflow valve 312, including a biological heart valve prosthesis, a mechanical heart valve prosthesis and a bio-mechanical heart valve prosthesis.

The valve 312 can include one or more valve members or leaflets 314 that are moveable to provide for substantially unidirectional flow of blood through the valve and into the interior chamber of the pouch 304. The valve 312 can also include an implantation flange (or sewing ring) 314 to facilitate securing the valve at an annulus (e.g., the atrioventricular annulus) of a patient's heart. The implantation flange 316 can be formed of a fabric material, a biological material, such as animal pericardium or a collagen web, or a combination of fabric and biological materials (e.g., a fabric sewing ring covered with biological tissue material).

As depicted, the heart valve 312 may be a biological heart valve prosthesis, such that only biological material is exposed. For example, the valve 312 can be a type of valve as shown in described in U.S. Pat. No. 6,610,088, which is entitled “BIOLOGICALLY COVERED HEART VALVE PROSTHESIS” the specification of which is incorporated herein by reference. Accordingly, the implantation flange 316, sidewall 310 and leaflets 314 thus can all comprise biological tissue material. Other types of heart valves and prostheses can also be used as well as various different types of materials to form a suitable heart valve prosthesis.

The pouch 304 has an interior chamber that defines a volume that can be filled (e.g., partially or fully) with blood. The inflow conduit 302 is in fluid communication with the interior chamber of the pouch 304 such that the valve 312 provides for substantially unidirectional flow of blood into the pouch. The pouch 304 can be considered generally spherical or ellipsoidal in shape when filled with fluid. The pouch 304 can be formed of a compliant biocompatible material. For example, the pouch can be formed of one or more sheets of a biological or a synthetic material, such as a natural tissue material (e.g., animal pericardium, dura matter) or a manufactured material (e.g., a collagen web).

In the example of FIGS. 7 and 8, the pouch 304 is formed from two generally calotte-shaped members 320 that have been attached together to define the interior chamber. Each calotte-shaped member 320 can be formed similar to the approach disclosed in U.S. Pat. No. 6,783,556, which is entitled “SYSTEM AND METHOD FOR MAKING A CALOTTE-SHAPED IMPLANTABLE SHEATH” and which is incorporated herein by reference. Other approaches can also be utilized to provide generally-calotte shaped members. By calotte-shaped, it is meant that the members 320 can be considered generally semi-spherical or semi-ellipsoidal, such that when the perimeters of the respective members are connected together they form a structure having an inner chamber that defines a desired volume, such as depicted in FIGS. 7 and 8. For example, when the pouch 304 is implanted in the ventricle, it provides means for limiting a volume of blood received within an enlarged ventricle of the patient's heart. The size and configuration of the pouch 304 can vary for a given application depending on, for example, the size of the patient's heart, the desired and the age of the patient as well as other circumstances and conditions of the patient.

The apparatus 300 also includes an outflow conduit 330 that is in fluid communication with the interior chamber of the pouch 304. The outflow conduit 330 extends from the pouch 304 and terminates in an outflow annulus 332 that is spaced apart from the pouch 304. In the example of FIGS. 7 and 8, an end 336 of the outflow conduit 330 is attached (e.g., by sutures) to a corresponding opening in the sidewall of the pouch 304. The outflow conduit 330 permits substantially free flow of fluid from the interior chamber of the pouch 304 and through the outflow conduit. For example, when the outflow end is located in a patient's aorta, the outflow conduit 330 provides means for providing a path for the flow of blood from within the pouch and into the aorta.

The outflow conduit 330 can be formed of a biological or synthetic material. For example, the outflow conduit can be formed from one or more sheets of a biological or a synthetic material, such as a natural tissue material (e.g., animal pericardium, dura matter) or a manufactured material (e.g., a collagen web). As an example, a sheet of treated animal pericardium (or other material) can be folded about a central longitudinal axis 338 and its opposed ends can be connected together (e.g., by sutures 334) and the folded sheet can be fixed and substantially detoxified to form the conduit 330.

The outflow conduit 330 can extend outwardly from the pouch 304 so that the longitudinal axis 338 thereof is substantially transverse to an exterior surface of the pouch. Similarly, the inflow conduit 302 can extend outwardly from another part of the pouch 304 so that a central longitudinal axis 340 of the inflow conduit is substantially transverse to an exterior surface of the pouch. By way of further example, the longitudinal axis 340 of the inflow conduit 302 and the longitudinal axis 338 of the outflow conduit 330 can define an angle 342 that is generally acute (e.g., less than about 90 degrees). Alternatively, the inflow and outflow conduits 302 and 330 can be connected to the pouch 304 so that other angles are formed by the respective longitudinal axes 340 and 338 in accordance with an aspect of the present invention.

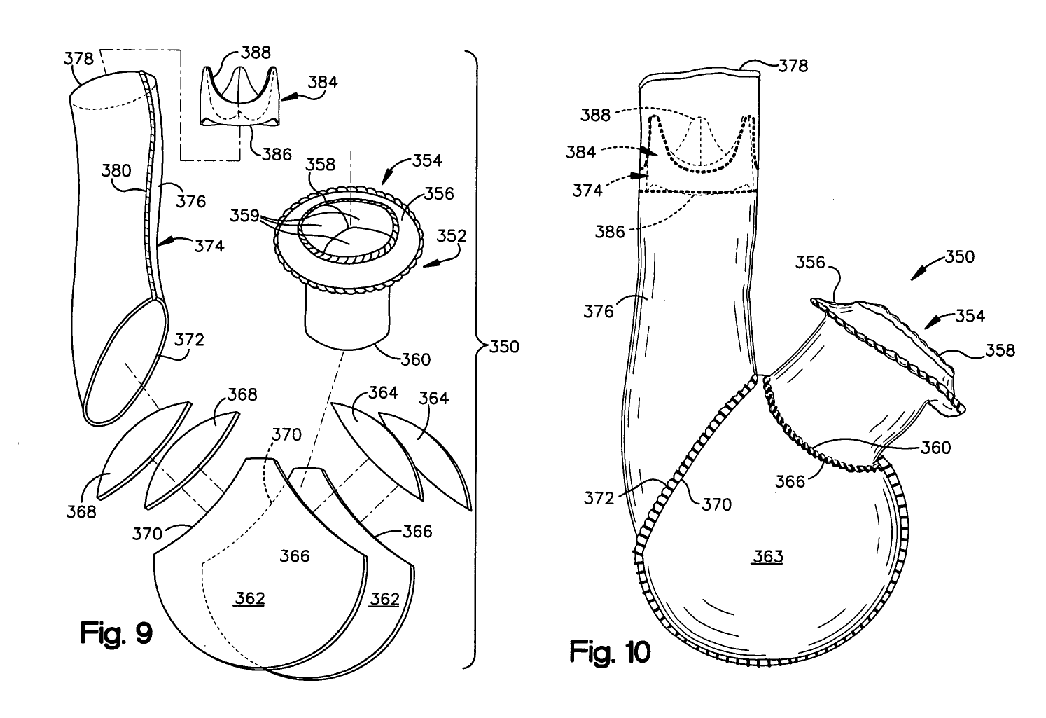

FIG. 9 depicts an example of an assembly view of an apparatus 350 that can be constructed according to an aspect of the present invention. FIG. 10 depicts an example of the assembled apparatus 350. The apparatus 350 includes an inflow conduit 352 that includes a heart valve 354, such as heart valve prosthesis as described herein. An implantation flange 356 can be provided at the inflow end 358 of the valve 354 to facilitate its attachment at an appropriate annulus of the patient's heart. The valve 354 can include one or more leaflets (or other members) 359 that cooperate to provide for substantially unidirectional flow of blood from the inflow end 358 to an outflow end 360 of the conduit 352. For example, the leaflets 359 are moveable between open and closed conditions to permit the flow of blood through the inflow conduit 352.

A pair of pouch members 362 can be connected together to define a pouch 363 (see FIG. 10) that includes an interior chamber that a defines a volume. As shown in FIG. 9, the pouch members 362 can be generally calotte-shaped members arranged so that their concave surfaces face toward each other. A first edge portion 364 can be removed (e.g., by cutting) from each of the pouch members 362 to provide corresponding edges 366 on the respective pouch members. Thus, when the pouch members 362 are attached together, as shown in FIG. 10, the edges 366 form a generally circular or generally elliptical opening to which the outflow end 360 of the inflow conduit 352 can be attached. Similarly, a second edge portion 368 can be removed (e.g., by cutting) from each of the pouch members 362 that have been trimmed to provide corresponding edges 370 on the respective pouch members. Accordingly, when the pouch members 362 are attached together, the edges 370 form a generally circular or generally elliptical opening to which an inflow end 372 of an outflow conduit 374 can be attached. The respective edge portions 364 and 368 can be removed before or after the pouch members 362 have been connected together.

The outflow conduit 374 can include a cylindrical sidewall portion 376 extending between the inflow end 372 and an outflow end 378. For example, the outflow conduit 374 can be formed from a sheet of a substantially biocompatible material by attaching opposed side edges together, such as by a suture line 380. The inflow end 372 can be cut on an angle relative to cylindrical sidewall portion 376 to provide a desired size opening (e.g., which can be larger than the transverse cross-section of the cylindrical portion 376) for attaching to the edges 370 of the pouch members 362.

The apparatus 350 further includes a second valve 384 that can be operatively associated with the outflow conduit 374 for providing for substantially unidirectional flow of blood through the outflow conduit. For example, the valve 384 can be located within and attached to the sidewall portion 376 of the outflow conduit 374, such as at an axial position that is between the inflow end 372 and the outflow ends 378. As an example, the valve 384 can be attached to the sidewall portion 376 at an axial position that is adjacent to the inflow end 372. However, the position of the valve 384 relative to the ends 372 and 378 can vary. For instance, the valve 384 may be affixed to the sidewall portion 376 after an appropriate position has been determined based on the size and anatomical geometry of the patients heart, which can be performed by imaging methods or actual measurements made during an implantation procedure.

The valve 384 includes an inflow end 386 that is spaced apart from an outflow end 388 by a sidewall portion 390. The heart valve 384 can be any type of heart valve prosthesis, such as a biological heart valve prosthesis, a mechanical heart valve prosthesis and a bio-mechanical heart valve prosthesis. The outflow end 388 can be configured to be generally sinusoidal, having sinuses between axially extending posts, as depicted in FIG. 9. Alternatively, the outflow end may have other configurations, such as a generally annular. As one example, the valve 384 can be a stentless natural tissue heart valve prosthesis. For the example of a natural tissue heart valve prosthesis (e.g., stented or unstented), the valve 384 can include one or more leaflets that are moveable relative to each other and the sidewall portion 390 to provide for the substantially unidirectional flow of blood. The particular mechanism for providing for the substantially unidirectional flow of blood through the valve will depend on the type of the valve that has been selected for use in the apparatus 350.

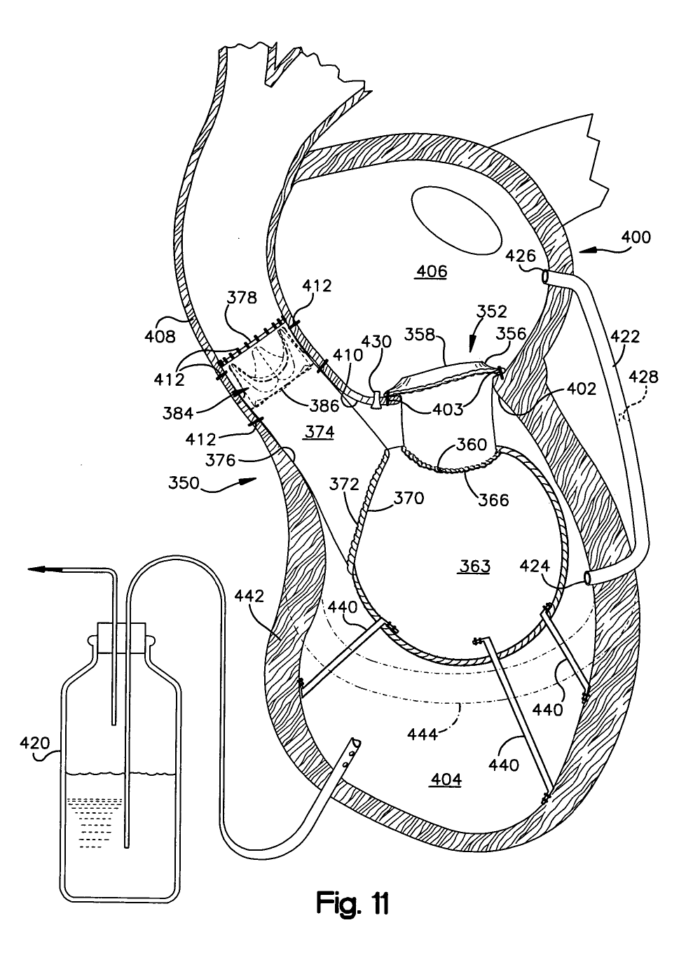

FIG. 11 depicts an example of the apparatus 350 of FIG. 10 implanted in a patient's heart 400 for improving ventricular function of the heart. For sake of brevity, the same reference numbers for the apparatus 350 are depicted in FIG. 11, and further information about such features can be had by way of reference to the preceding description herein. Prior to implanting the apparatus 350, the patient's own aortic and mitral valves (or at least calcified portions thereof) should be removed.

As shown in the example of FIG. 11, the valve 354 is secured at the atrioventricular annulus 402 of the heart 402. For instance, the implantation flange 356 can be secured by a continuous suture 403 (or other means) at the atrial side of the annulus 402. The inflow conduit 352 and the pouch 363 extend from the attachment at the annulus 402 into the left ventricle 404. The valve 354 thus permits unidirectional flow of blood from the left atrium 406 into the pouch 363.

The outflow conduit 374 is positioned within the aorta 408. The outflow valve 384 is located near the aortic annulus 410. The outflow valve 384 can be attached to the sidewall portion 376 of the outflow conduit 374 prior to implanting the apparatus 350 in the ventricle 404 or it can be attached during the implantation procedure (e.g., before the apparatus has been attached within the heart 400). The sidewall portion 376 of the outflow conduit 374 can be attached to the aorta 408 by sutures 412, although other attachment mechanisms can be use separately or in addition to the sutures. Since the outflow valve 384 is affixed within the outflow conduit 374, the valve becomes affixed within the aorta 408 when the sidewall portion 376 is secured relative to the aorta. The outflow valve 384 thus provides for substantially unidirectional flow of blood from within the interior chamber of the pouch 363 into the aorta 408 in response to contraction of the ventricle 404 by associated cardiac muscle 442. That is, the contraction of the ventricular cardiac muscle causes the blood from the interior chamber to be forced through the valve 384 and into the aorta 408, while the inflow valve 354 prevents regurgitation (or backflow) into the atrium 406.

Additionally, to facilitate implantation of the apparatus 350 within the ventricle 404, a vacuum assembly or pump 420 can be employed to remove fluid from the patient's dilated ventricle similar to as described above with respect to FIG. 5. By removing the blood from the dilated ventricle 404, self-remodeling of the cardiac muscle to a more normal size is facilitated. The pump 420 would be removed after the implantation has been completed and most (if not all) blood has been removed from the space between the apparatus 350 and ventricular cardiac muscle 442.

Additionally or alternatively, one or more conduits can be utilized to provide a path for the flow of blood from the ventricle 404 into the atrium 406. By way of example, an external conduit 422 can be implanted with a first end 424 located in the ventricle 404 and a second end 426 located in the atrium 426. The conduit 422 can include one or more valves 428, such as biological valves (e.g., venous valves, small heart valve prostheses), mechanical valves, or other types of valve devices to provide for substantially unidirectional flow of blood from the ventricle 404 to the atrium 406. As a result, any blood remaining in the ventricle 404 thus can be urged through the conduit 422 and into the atrium 406 during subsequent cardiac cycles, so that the blood re-enters circulation. The conduit 422 can be a synthetic material (e.g., polymer) or a biological material, such as a natural tissue (e.g., a vein or artery or a sheet of natural tissue formed into the conduit) or processed biological material (e.g., a collagen-like tube).

As another example, as small internal conduit 430 can be attached in the heart between the ventricle 404 and the atrium 406, such as through tissue that forms is located near to the atrioventricular annulus 402. The conduit 430, for example, can be secured at the annulus 402 when the heart valve 354 is secured at the annulus, as described above. The conduit 430 can be a short conduit (e.g., a catheter or shunt apparatus) that having a greater number of openings in the ventricular side than in the atrial side so that the increased pressure in the ventricle 404 causes blood from the ventricle to flow through the conduit 430 and into the atrium 406. Other types of conduits with or without valves, which can be made of various types of biocompatible materials, can also be utilized. It is to be understood that the conduits 422 and 430 can also be utilized with any of the approaches described herein, including but not limited to FIGS. 5 and 6.

Additionally, as with the approaches described above (FIGS. 5 and 6), tethers 440 can be attached between the pouch 363 and the surrounding cardiac muscle 442 of the ventricle 404. The tethers 440 thus can help hold the pouch 363 in a desired configuration, as described herein.

The interstitial space in the ventricle 404 between the pouch 363 and the cardiac muscle 442 will reduce over time, enabling the heart to self-remodel and function more normally. The remodeling can be facilitated by removing surrounding fluid, such as via suction device 420 as well as (or alternatively) by employing one or more conduits 422 and 430. For example, the cardiac muscle 442 will self-remodel over time and return the heart to a reduced size, as depicted in dashed lines at 444. In view of the foregoing, those skilled in the art will understand and appreciate that the approaches described herein can be employed to significantly improve ventricular function.

What has been described above includes examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

1. An implantable apparatus, comprising:
an inflow conduit having first and second ends spaced apart from each other by a sidewall portion;
an inflow valve operatively associated with the inflow conduit to provide for substantially unidirectional flow of blood through the inflow conduit from the first end to the second end of the inflow conduit;
a pouch having an interior chamber that defines a volume, the inflow conduit being in fluid communication with the interior chamber of the pouch; and
an outflow conduit in fluid communication with the interior chamber of the pouch to permit substantially free flow of fluid from the interior chamber of the pouch and into the outflow conduit, which terminates in an outflow annulus spaced from the pouch.

2. The apparatus of claim 1, wherein each of the pouch, the inflow conduit, and the outflow conduit comprises a biological material.

3. The apparatus of claim 1, wherein, the second end of the inflow conduit is connected to the pouch and the outflow conduit is connected to the pouch, each of the inflow conduit and the outflow conduit having a central longitudinal axis that is substantially transverse to an exterior surface of the pouch.

4. The apparatus of claim 3, wherein the central longitudinal axis of the inflow conduit and the central longitudinal axis of the outflow conduit define an angle that is generally acute.

6. The apparatus of claim 1, wherein the pouch comprises at least one sheet of a biological material configured to provide the interior chamber.

7. The apparatus of claim 6, wherein the at least one sheet of biological material further comprises a pair of substantially calotte-shaped members attached together near a perimeter thereof to provide the interior chamber.

8. The apparatus of claim 6, wherein the at least one sheet of biological material further comprises animal pericardium.

9. The apparatus of claim 1, further comprising an outflow valve operatively associated with the outflow conduit to provide for substantially unidirectional flow of blood from within the internal chamber of the pouch and through outflow conduit.

10. The apparatus of claim 9, wherein the outflow valve is located within the outflow conduit spaced from an end of the outflow conduit that is attached to the pouch.

11. The apparatus of claim 10, wherein the outflow valve further comprises one of a biological heart valve prosthesis, a mechanical heart valve prosthesis and a bio-mechanical heart valve prosthesis.

12. The apparatus of claim 1, wherein the wherein the inflow conduit defines a valve wall portion in which the inflow valve is located.

13. The apparatus of claim 1, wherein the inflow valve further comprises one of a biological heart valve prosthesis, a mechanical heart valve prosthesis and a bio-mechanical heart valve prosthesis.

14. An implantable apparatus for improving ventricular function, comprising:
means for limiting a volume of blood received within an enlarged ventricle of the patient's heart;
means for providing for substantially unidirectional flow of blood into the means for limiting;
means for providing a path for flow of blood from within the means for limiting and into an aorta of the patient's heart; and
means, located within the means for providing a path, for providing for substantially unidirectional flow of blood out of the means for limiting and into the aorta.

15. The apparatus of claim 14, further comprising means for tethering a portion of the means for limiting relative to cardiac tissue of the patient's heart so as to maintain a desired configuration of the means for limiting.

16. The apparatus of claim 14, wherein the means for limiting further comprises a pouch formed of at least one sheet of a biological material configured to receive a volume of blood in the interior chamber thereof.

17. A method for improving ventricular function of a heart, comprising:
implanting a pouch in a ventricle of the heart, the pouch including an interior chamber that defines a volume;
mounting an inflow valve at a mitral position of the heart, the inflow valve being in fluid communication with the interior chamber of the pouch to provide for substantially unidirectional flow of blood from an atrium of the heart through the inflow valve and into the interior chamber of the implanted pouch; and
attaching an outflow conduit, which is in fluid communications with the interior chamber of the implanted pouch, near an aortic annulus to provide for substantially unidirectional flow of blood from the interior chamber of the pouch and into the aorta of the heart.

18. The method of claim 17, wherein an outflow valve is operatively connected to the outflow conduit to provide for the substantially unidirectional flow of blood from the interior chamber of the pouch into the aorta.

19. The method of claim 17, further comprising tethering an exterior of the sidewall of the pouch relative to surrounding cardiac muscle.

20. The method of claim 17, further comprising removing blood from a space in the ventricle between the pouch and surrounding cardiac tissue to facilitate self-remodeling of the heart.

21. The method of claim 20, further comprising attaching at least one conduit between the ventricle and the atrium to provide a path for flow of blood from the space in the ventricle to the atrium.
